(19) United States
(12) Patent Application Publication
(10) Pub. No.: US 2001/0004692 A1
(43) Pub. Date: Jun. 21, 2001

(54) Endoscopic spraying instrument

(75) Inventors: Satoshi Kidooka, Tokyo (JP); Teruo Ouchi, Saitama (JP)

Correspondence Address: GREENBLUM & BERNSTEIN, 1941 ROLAND CLARKE PLACE, RESTON, VA, 20191

(73) Assignee: ASAHI KOGAKU KOGYO KABUSHIKI KAISHA

(21) Appl. No.: 09/735,551

(22) Filed: Dec. 14, 2000

(30) Foreign Application Priority Data
Dec. 15, 1999 (JP) P.HEI.11-355305

(51) Int. Cl. A61B 18/18; A61B 1/00

(52) U.S. Cl. 606/46; 600/139

(57) ABSTRACT

In an endoscopic spraying instrument, liquid passed through a liquid supplying tube and a rotatingly guiding groove disposed at a leading end side of the supplying tube is rotated about an central axis within a liquid rotating chamber disposed at a leading end side of the rotatingly guiding groove and discharged forwardly from an ejection hole formed in a leading end wall of the liquid rotating chamber. An annular, protruded wall, is provided, which is spaced outwardly from an outer periphery of the ejection hole, which is protruded forwardly, and which surrounds an exit of the ejection hole.

### BACKGROUND OF THE INVENTION

This invention relates to an endoscopic spraying instrument which is used to spray liquid within a body cavity by being passed through a treatment instrument channel of an endoscope.

FIG. 5 is a typical endoscopic spraying instrument, which includes a flexible liquid supplying tube 91 to be removably passed through a treatment insertion channel of an endoscope, and a screw member 92 arranged at a leading end opening of the liquid supplying tube 91. The screw member 92 is formed at its outer circumference with a spiral groove 93.

A leading end cap 94 covers the screw member 92 to close a radially outer side of the spiral groove 93, and a liquid rotating chamber 95 is defined inside the leading end cap 94 in front of the leading end surface of the screw member 92. The liquid is supplied through the liquid supplying pipe 91, and passed through the spiral groove 93 to reach the liquid rotating chamber 95 where the liquid is rotated about a longitudinal axis of the instrument.

A leading end wall of the liquid rotating chamber 95 is formed at its central position with an ejection hole 96, and a wall surface 97 around an exit of the ejection hole 96 is formed into a shallow concave shape taped in section.

With this arrangement, the liquid such as medicament liquid and chromogenic liquid, supplied forwardly through the liquid supplying tube 91 is forwardly ejected from the ejection hole 96 while being rotated in the liquid rotating chamber 96, thereby realizing a spraying state with a wide spraying angle.

It is preferable for the endoscopic spraying instrument to spray the liquid in any directions uniformly. However, the spraying amount maybe largely varied depending on the directions, as shown in FIG. 5, due to processing error or assembling error of the screw member 92 or other components, or the presence of small dusts in the liquid passage

Further, as shown in FIG. 6, a part of the ejected liquid may flow on and along the outer surface of the leading end cap 94, making the spraying state unstable.

### SUMMARY OF THE INVENTION

Accordingly, an object of the present invention is to provide an endoscopic spraying instrument, which can spray liquid uniformly over all directions.

According to the present invention, an annular, protruded wall is formed, which surrounds an exit of an ejection hole. Consequently, even if the amount of liquid ejected from the ejection hole is largely varied depending on the ejecting directions, a part of the ejected liquid, which is large in ejection amount and deviates outwardly, collides against and thus is reflected by the annular protruded wall, so that the spraying amount in each direction can be uniform or averaged. Thus, the uniform spraying of the liquid to the exterior can be realized.

Preferably, the present invention is applied to an endoscopic spraying instrument in which liquid passed through a liquid supplying tube and a rotatingly guiding groove disposed at a leading end side of the supplying tube is rotated about an central axis within a liquid rotating chamber disposed at a leading end side of the rotatingly guiding groove and discharged forwardly from an ejection hole formed in a leading end wall of the liquid rotating chamber. In the instrument, an annular, protruded wall is provided, which is spaced outwardly from an outer periphery of the ejection hole, which is protruded forwardly, and which surrounds an exit of the ejection hole.

A wall surface extending between the outer periphery of the ejection hole and the annular, protruded wall may be defined by a tapered surface or a curved, concave surface, or may be defined by a planar surface perpendicular to an axis of the ejection hole.

A wall surface of the annular, protruded wall may be parallel to an axis of the ejection hole or may be defined by a forwardly spread surface or a forwardly constricted surface.

The present disclosure relates to the subject matter contained in Japanese patent application No. Hei. 11-355305 (filed on Dec. 15, 1999), which is expressly incorporated herein by reference in its entirety.

### BRIEF DESCRIPTION OF THE DRAWINGS

FIG. 1 is a longitudinal sectional view showing a leading end part of an endoscopic spraying instrument according to a first embodiment of the present invention.

FIG. 2 is a perspective view showing an entire structure of the endoscopic spraying instrument according to the first embodiment of the present invention.

FIG. 3 is a longitudinal sectional view showing a leading end part of an endoscopic spraying instrument according to a second embodiment of the present invention.

FIG. 4 is a longitudinal sectional view showing a leading end part of an endoscopic spraying instrument according to a third embodiment of the present invention.

FIG. 5 is a longitudinal sectional view showing a leading end part of a typical endoscopic spraying instrument.

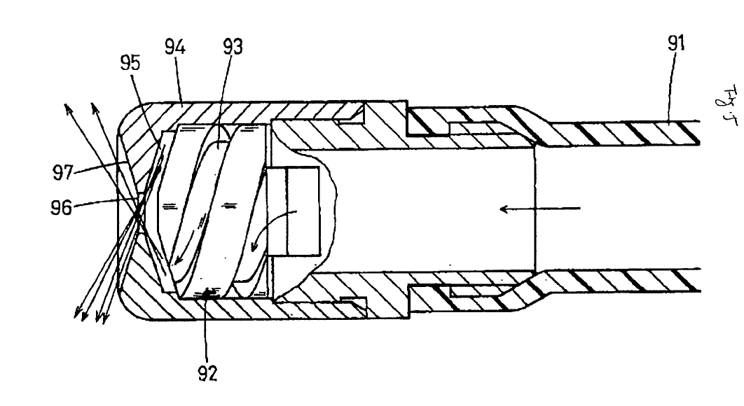

FIG. 6 is a longitudinal sectional view showing the leading end part of the typical endoscopic spraying instrument.

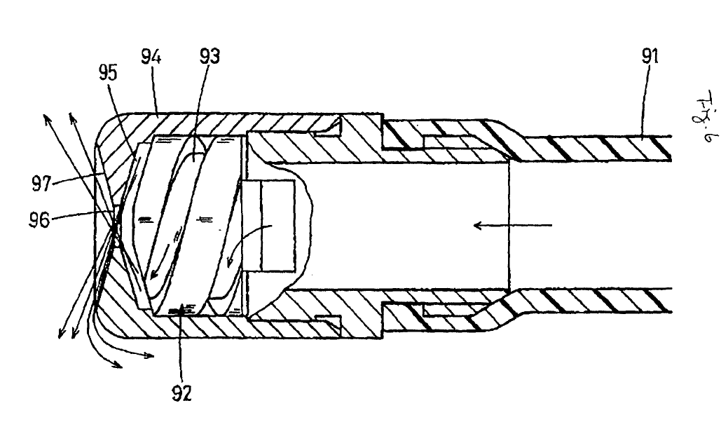

### DESCRIPTION OF THE PREFERRED EMBODIMENT

Preferred embodiments of the present invention will be described with reference to the accompanying drawings.

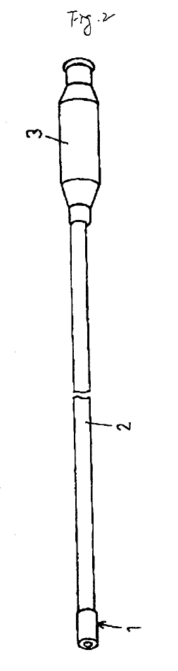

FIG. 2 shows an endoscopic spraying instrument according to a first embodiment of the present invention, which includes a liquid supplying tube 2, such as a flexible tube of tetrafluoroethylene resin, removably insertable into an unillustrated treatment instrument insertion channel of an endoscope, and a leading end nozzle 1 attached to a leading end of the liquid supplying tube 2.

Connected to the trailing end of the liquid supplying tube 2 is an injection joint 3 to which a unillustrated syringe or the like can be connected so that liquid such as medicament liquid and chromogenic liquid is supplied through the liquid supplying tube 2 into the leading end nozzle 1.

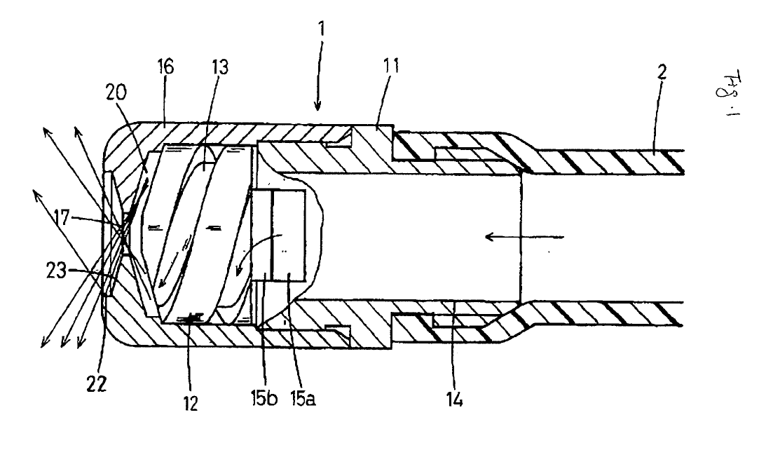

FIG. 1 shows a structure of the leading end nozzle 1. A male thread formed on the rear end outer circumference of a nozzle main body 11 is threadingly inserted into the leading end of the liquid supplying tube 2 and adhered thereto so that the nozzle main body 11 is fixedly coupled to the leading end of the liquid supplying tube 2.

A screw body 12, formed at it outer circumference with a spiral groove (rotatingly guiding groove) 13, is disposed adjacent to the leading end side of the nozzle body 11. The spiral groove 13 may be formed by a plurality of grooves or otherwise a single groove.

The nozzle main body 11 has a flow passage hole 14 which is concentric to a longitudinal axis thereof and is passed therethrough. The flow passage hole 14 extends from the rear end of the nozzle main body 11, and is communicated through a communication hole 15a and a groove 15b, both being formed at the leading end side of the nozzle main body 11, with the spiral groove 13.

A leading end cap 16 is fixedly fitted to the front half of the nozzle main body 11 such that the screw member 12 is interposed and clamped therebetween and the radially outer side of the spiral groove 13 is closed. Consequently, the spiral groove 13 is constructed as such a closed groove that the radially inner and outer sides are closed but the leading and trailing end are open.

A liquid rotating chamber 20 is formed between the leading end wall of the nozzle main body 11 (the leading end wall of the screw member 12) and the leading end wall of the leading end cap 16, and the leading end of the spiral groove 13 is open to the liquid rotating chamber 20. Therefore, the liquid, passed through the spiral groove 13 and discharged from the leading end thereof, are rotated about a longitudinal axis within the liquid rotating chamber 20. An ejection hole 17 is formed through the leading end wall of the leading end cap 16 at a central position thereof. The diameter of the ejection hole is, for example, about 0.4 mm to 0.6 mm.

The rear side inner wall surface of the liquid rotating chamber 20 (i.e. a leading end wall surface of the screw member 12) and the leading end side inner wall surface thereof are formed as conical surfaces that are convex toward the ejection hole 17 and that are spaced from each other at substantially constant distance.

The leading end cap 16 is formed at its leading end surface with an annular protruded wall 22, which is spaced outwardly from the outer periphery of the ejection hole 17, which is protruded forwardly (in a direction parallel to an axis of the ejection hole 17 in this embodiment) and which surrounds an exit of the ejection hole 17. The protruded height of the annular protruded wall 22 is, for example, about 0.04 mm to 0.3 mm.

A tapered, conical wall surface 23 is defined, extending between the outer periphery of the ejection hole 17 and the annular protruded wall 22. An angle of the wall surface 23 with respect to the axis is, for example, about 45 degrees to 120 degrees.

With this arrangement, the liquid, such as medicament liquid and chromogenic liquid, supplied forwardly through the liquid supplying tube 2 is passed through the spiral groove 13 so that the liquid is forwardly ejected (sprayed) from the ejection hole 17 while being rotated in the liquid rotating chamber 20.

Even if the amount of liquid ejected from the ejection hole 17 is largely varied depending on the ejecting directions, a part of the ejected liquid, which is large in ejection amount and deviates outwardly, collides against and thus is reflected by the annular protruded wall 22, thereby being dispersed appropriately. Consequently, the spray of the ejected liquid is made uniform. Further, there occurs no liquid flow rearwardly flowing on and along the outer surface of the leading end cap 16.

Accordingly, the amount of liquid ejection in each direction can be made uniform or averaged and the uniform spray of the liquid can be realized. The experimental result showed a remarkable advantage obtained by this structure. That is, without strict control of dimensional precision for the screw member 12 and other components, the spraying amount was uniform over the all directions and the rearward flow of the liquid on and along the outer surface of the leading end cap 16 was not found.

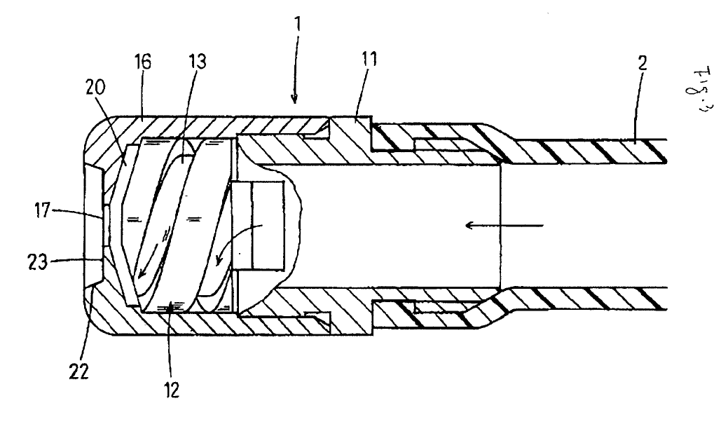

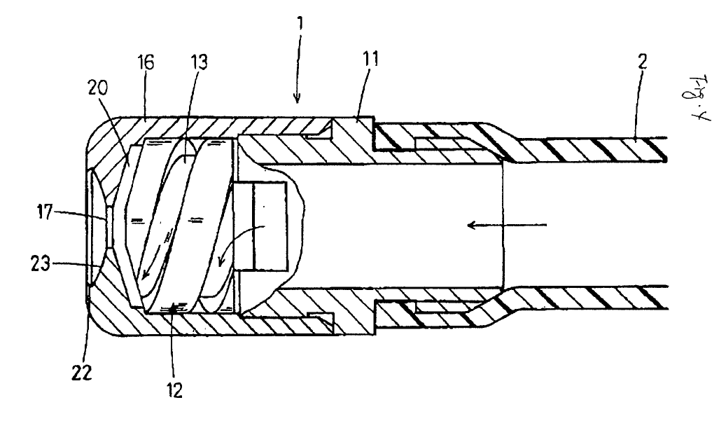

In addition, the present invention should not be restricted to the aforementioned embodiment. For example, as shown in FIGS. 3 and 4, the wall surface of the annular protruded wall 22 may spread or constricted forwardly, and further, the wall surface 23 extending between the outer periphery of the ejection hole 17 and the annular protruded wall 22 may be planar or curved (concave).

The groove which gives a rotating force to the liquid should not be restricted to the spiral groove 13, and may be formed as a groove having any arbitrary shape including a linear shape. The rotating chamber 20 may also be formed to have any arbitrary shape.

What is claimed is:

1. An endoscopic spraying instrument in which liquid passed through a liquid supplying tube and a rotatingly guiding groove disposed at a leading end side of the supplying tube is rotated about an central axis within a liquid rotating chamber disposed at a leading end side of the rotatingly guiding groove and discharged forwardly from an ejection hole formed in a leading end wall of the liquid rotating chamber, the instrument comprising:
an annular, protruded wall, which is spaced outwardly from an outer periphery of the ejection hole, which is protruded forwardly, and which surrounds an exit of the ejection hole.

2. The instrument of claim 1, wherein an wall surface extending between the outer periphery of the ejection hole and the annular, protruded wall is defined by a tapered surface or a curved, concave surface.

3. The instrument of claim 1, wherein an wall surface extending between the outer periphery of the ejection hole and the annular, protruded wall is defined by a planar surface perpendicular to an axis of the ejection hole.

4. The instrument of claim 1, wherein a wall surface of the annular, protruded wall is parallel to an axis of the ejection hole.

5. The instrument of claim 1, wherein a wall surface of the annular, protruded wall is defined by a forwardly spread surface or a forwardly constricted surface.

6. A cap member to be provided to a leading end of an endoscopic spraying device, the cap member comprising:
a cylindrical portion defining an interior of the cap member; and
a leading end wall at a leading end of the cylindrical portion, the leading end wall separating the interior of the cap member from an exterior thereof, the leading end wall having an ejection hole communicating the interior with the exterior, the leading end wall including:
a first wall surface in the exterior of the cap member, the first wall surface extending radially outwardly from an outer periphery of the ejection hole; and
a second wall surface in the exterior of the cap member, the second wall surface extending longitudinally outwardly from an outer periphery of the first wall surface.

7. The cap of claim 6, wherein the first wall surface is conical.

8. The cap of claim 7, wherein the first wall surface is curved in section.

9. The cap of claim 7, wherein the first wall surface is planar in section.

10. The cap of claim 6, wherein the first wall surface is planar.

11. The cap of claim 6, wherein the second wall surface extends radially inwardly from the periphery of the first wall surface.

12. The cap of claim 6, wherein the second wall surface extends radially outwardly from the periphery of the first wall surface.
